(19) United States
(12) Patent Application Publication
(10) Pub. No.: US 2011/0125133 A1
(43) Pub. Date: May 26, 2011

(54) Catheter Components and Assembly

(75) Inventors: Steen Aggerholm, St. Heddine (DK); Scott K. Philhower, Bloomington, IN (US)

(73) Assignees: Sabin Corporation, Bloomington, IN (US); William Cook Europe ApS, Bjaeverskov (DK)

(21) Appl. No.: 12/677,058

(22) PCT Filed: Sep. 18, 2008
(86) PCT No.: PCT/US08/76882

§ 371 Date: Dec. 9, 2010

Related U.S. Application Data
(60) Provisional application No. 60/994,772, filed on Sep. 21, 2007.

(51) Int. Cl. A61M 39/10

(52) U.S. Cl. 604/523

(57) ABSTRACT

A catheter assembly is provided with a stress relief collar for relieving stress due to differences in rigidities of a catheter and coupled fitting. The stress relief collar is provided with an integral clip of the same or a similar soft material. The catheter can be coiled and held in the clip, the flexibility of the latter making it easy to fit and remove coils therefrom with little risk of damage to the catheter.

### FIELD OF THE INVENTION

The present invention relates to components of a medical introducer, such as a catheter, and to an introducer assembly for use in endoluminal deployment and medical treatment devices.

### BACKGROUND OF THE INVENTION

A typical endoluminal deployment system includes an elongate carrier element, typically a catheter, cannula, pusher member or the like, for carrying a medical device to be implanted in a patient. The implant might be a stent, a stent graft, a filter, an occlusion device or any other implantable device of such a nature.

Many deployment systems also include a guide wire which is the first element inserted into the vasculature of the patient during a deployment procedure. Being very flexible, the guide wire is able to move along the tortuous vascular paths of a patient relatively easily until its distal end reaches the site at which an implant is to be deployed or, in the case of a treatment device, the location at which treatment is to be effected. Once so positioned, the guide wire provides a guide which the other components follow to the deployment or treatment site. This is typically achieved by providing these other components with an internal bore into which the guide wire can slide.

In the case of deployment of an implant, when the elongate carrier has been correctly inserted, the device is released and deployed in the desired position. The deployment operation can involve any one or more of several well known procedures, such as retraction of the covering sheath, release of restraining wires and so on.

Typically, in the operating theatre the medical staff will prepare and make ready for use the various components of the deployment device at the appropriate time. In particular, the staff will unpack the device holding catheter or element from its packaging so that it can be immediately available to the surgeon. Such catheters can be long, sometimes greater than one metre. As a result of their length, they are typically kept in a coiled state and uncoiled only when they are to be inserted into the patient. The simplest method of keeping such catheters coiled is to place them on a clean surface with a weight on top of the turns of coil. This is, however, a highly unsatisfactory solution in part because it is unreliable. In particular, the catheter is liable to uncoil as a result of its springiness and thus its natural tendency to do so. It may also uncoil if the overlying weight is accidentally dislodged. The springiness of the catheter has been known to cause it to jump from its resting position, and in some instances to fall on the floor. Should this occur, the catheter and implant device held thereon become unusable because of contamination.

There are known in the art other mechanisms for holding a catheter in a coiled configuration until its moment of use.

For example, U.S. Pat. No. 6,290,691 discloses a clip arrangement for temporarily storing a catheter device in a looped or coiled configuration in an operating theatre. The clip arrangement is part of the hub of the catheter assembly, which includes a fitting for a luer lock. The clip includes a lid hinged to the hub and a snap-fitting clasp which engages a catch on the hub body. In order to hold a catheter shaft in a coiled configuration, the clasp lid is opened so as to allow the coils of catheter shaft to be located in the recess of the hub and the clasp lid is then latched closed. The catheter shaft can be removed by unhooking the catch and pivoting this to its open position.

U.S. Pat. No. 5,372,592 discloses a tool which is used in the handling and flushing of the guide wire lumen of a catheter prior to its use in an angioplasty procedure. The tool includes a luer fitting to which there is integrally formed a clip which is described as engaging the body of the catheter in several locations along its length. The clip includes a pair of spaced part fingers which may be made from the same material as the luer fitting. A plurality of coil loops of the catheter may be held within the clip.

U.S. Pat. No. 5,769,222 discloses a variety of holders for holding a catheter in a coiled configuration. In one embodiment a plurality of clips are provided on a base member.

U.S. Pat. No. 6,047,825 discloses a coiled tubular holder for storing a plurality of guide wires in which the guide wires extend from a flared end cone of the coiled holder. The tubing of the holder is kept in its coiled configuration by a plurality of hook elements which are designed to hold the tubing and not the guide wire or any catheter.

U.S. Pat. No. 3,049,946, which is directed to a different technical field unrelated to medical devices, discloses a strain relief sleeve for an electrical wire which includes an integral chuck key holder. This is designed specifically for an electric drill. The strain relief sleeve is of the type which is intended to hold a mains electricity cable firmly therewithin so that the cable cannot be pulled out of the component when the cable is pulled and is not intended for providing radial strain relief. Furthermore, the clip for the chuck key requires insertion of the chuck key from below in a direction longitudinal to the bore of the clip.

It is also known to provide catheter clips formed within the rigid carrier of the catheter assembly which also provides the luer lock or other fitting.

It is believed that the rigid clips are intended to provide a firm grip of the catheter coils. While these can work satisfactorily with some types of catheters, they can also provide difficulties including significant effort in fixing a catheter therein or removing the catheter coil at the time of use. Moreover, these clips can also damage thin or weak catheters, particularly by kinking these during the insertion or removal processes. If the catheter coil kinks, it becomes useless and the entire catheter assembly must be discarded.

### SUMMARY OF THE INVENTION

The present invention seeks to provide an improved catheter assembly, an improved catheter component and an improved catheter stress relief fitting.

According to an aspect of the present invention, there is provided a fitting for a medical introducer assembly including an elongate collar made from resilient material and a clip element integral with the elongate collar and extending outwardly therefrom, the clip being made from a material having substantially the same flexibility as the material of the elongate collar.

Advantageously, the fitting is a stress relief fitting.

Preferably, the clip is made from the same material as the elongate collar.

It is envisaged that the medical introducer assembly may be a catheter assembly.

A flexible catheter holding clip represents a departure from the above-mentioned prior art disclosures which all provide for a substantially rigid clip. It has been found that a clip made from a pliant material can provide sufficient holding force to hold reliably one or a plurality of turns of a catheter and yet substantially facilitates the insertion and retraction of the turns from the clip. This is particularly advantageous in the case of thin catheters which are prone to being damaged by excessive manipulation or when forced into or out of holding components. The term substantially the same flexibility is therefore intended to encompass materials which are substantially softer than the rigid fitting and preferably softer than the catheter.

Advantageously, the clip includes first and second facing fingers providing therebetween a space for receiving a section of catheter. Preferably, the fingers include at their ends remote from the collar an elongate opening for the passage of a section of catheter therethrough into the space. It is preferred that the fingers provide an elongate opening which is narrower than the space. In one embodiment, this is achieved by providing at the remote ends of the fingers enlarged facing feet members. The space may be rounded in longitudinal cross-section.

This embodiment has the advantage of providing what is in effect an enlarged space between the fingers within which the catheter coil sections are held. The feet assist in holding the coil sections within the clip reliably until the time of deployment and with the benefits of the pliability of the fingers. The elongate opening allows the catheter sections to be inserted into and removed from the clip by means of a radial movement towards and outwardly from the clip and thus of the stress relief collar.

It is preferred that the clip extends only part way along the stress relief collar and, more preferably, is located on a proximal position of the collar. In one embodiment, the clip has a length of 2 centimetres or less, more preferably 1 centimetre or less.

Advantageously, the fingers of the clip taper towards the collar in the distal direction thereof.

The clip may be formed of any of the materials used for strain relied sleeves including but not limited to silicon, silicone rubber, nylon, Pebax™ (polyester bloc amide).

Preferably, the clip is formed of a material having a hardness on the Shore D scale of between 20 and 50, more preferably between 30 and 40. In one embodiment, the clip is made from a material having a hardness of 38 D on the Durometer Scale.

According to another aspect of the present invention, there is provided a medical introducer assembly including an elongate introducer element, a fitting attached to the elongate introducer element, a flexible collar and a clip for holding one or more sections of the elongate introducer element, the clip being formed of a material having a flexibility no greater than the flexibility in curvature of the introducer element.

According to another aspect of the present invention, there is provided a catheter assembly including an elongate catheter, a fitting attached to the elongate catheter, a flexible collar and a clip for holding one or more sections of the elongate catheter, the clip being formed of a material having a flexibility no greater than the flexibility of the catheter.

Preferably, the collar is a stress relief collar for relieving stress differentials between the fitting and the elongate catheter.

Advantageously, the clip is formed of a material substantially softer than the catheter, so that the catheter can be pushed into and pulled out of the clip with little risk of damage thereto.

The stress relief collar is preferably located over the elongate catheter and connected to the fitting. In one embodiment, the stress relief collar is located over a distal end of the fitting.

Preferably, the clip is integral with the stress relief collar.

Advantageously, the clip is made of the same material as the stress relief collar.

### DESCRIPTION OF THE DRAWINGS

Embodiments of the present invention are described below, by way of example only, with reference to the accompanying drawings, in which:

FIGS. 1 and 2 are perspective views of an example of known stent delivery device which can be modified to include a catheter assembly according to the teachings herein;

FIG. 3 is a view of an embodiment of catheter assembly;

FIG. 4 is an end elevational view of a stress relief collar of the assembly of FIG. 3;

FIG. 5 is a view of the catheter assembly of FIG. 3 showing a catheter element coiled and held by a holding clip of the assembly; and

FIG. 6 is a side elevational view of another embodiment of catheter assembly.

### DESCRIPTION OF THE PREFERRED EMBODIMENTS

It is to be understood that the Figures are schematic and do not show the various components in their actual scale. In many instances, the Figures show scaled up components to assist the reader.

In this description, when referring to a deployment assembly, the term distal is used to refer to an end of a component which in use is furthest from the surgeon during the medical procedure, including within a patient. The term proximal is used to refer to an end of a component closest to the surgeon and in practice in or adjacent an external manipulation part of the deployment of treatment apparatus.

On the other hand, when referring to an implant such as a stent or stent graft, the term proximal refers to a location which in use is closest to the patient's heart, in the case of a vascular implant, and the term distal refers to a location furthest from the patient's heart.

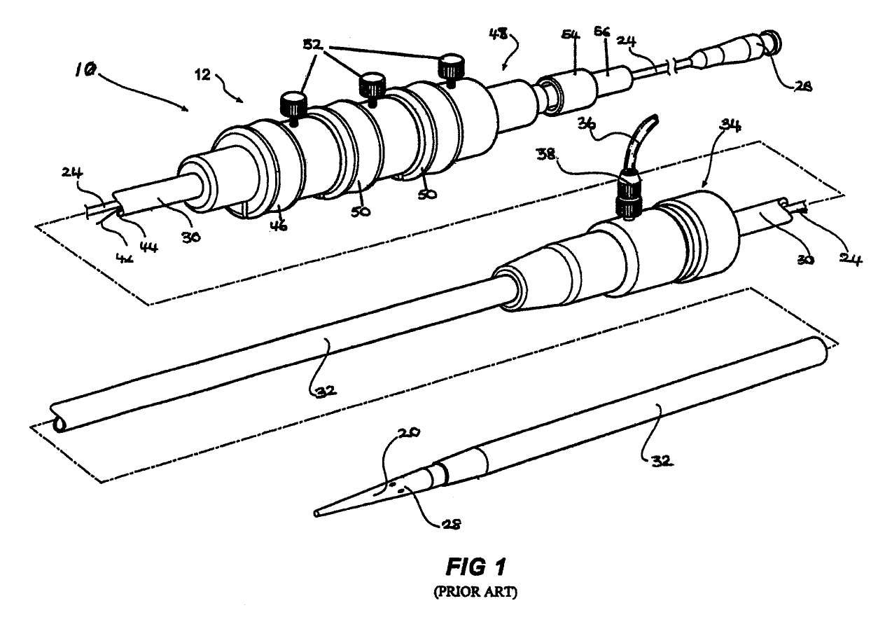

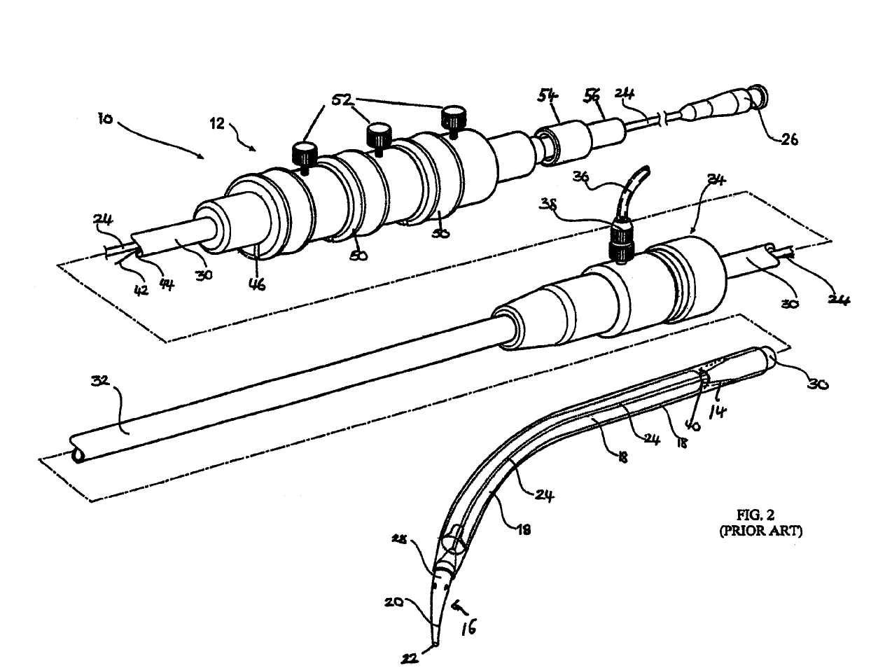

Referring to FIGS. 1 and 2, the introducer 10 includes an external manipulation section 12, a proximal attachment region 14 and a distal attachment region 16. The proximal attachment region 14 and the distal attachment region 16 secure the two ends of the implant 18. During the medical procedure to deploy the implant 18, the proximal and distal attachment regions 14 and 16 will travel through the patient's vasculature, in this example, to a desired deployment site. The external manipulation section 12 at the proximal end of the assembly 10, which is operated by a surgeon to manipulate the introducer, remains outside of the patient throughout the procedure.

The distal attachment region 16 of the introducer 10 includes a dilator tip 20, which is typically provided with a bore 22 therein for receiving a guide wire (not shown) of conventional type. The longitudinal bore 22 also provides a channel for the introduction of medical reagents. For example, it may be desirable to supply a contrast agent to allow angiography to be performed during placement and deployment phases of the medical procedure.

An inner catheter or cannula 24, conventionally made from a flexible thin walled metal tube, is fastened to the dilator tip 20. The inner catheter 24 is flexible so that the introducer 10 can be advanced along a relatively tortuous vessel, such as a femoral artery, and so that the distal end of the assembly 10 can be longitudinally and rotationally manipulated. The inner catheter 24 carries a stent 18 or other device to be implanted in the patient. The catheter 24 extends through the introducer 10 to the manipulation section 12, terminating at a connection device 26, in conventional manner.

The connection device 26 is designed to accept a syringe to facilitate the introduction of reagents into the inner catheter 24 and for this purpose is typically provided with a threaded luer lock connection.

Where provided, a pusher sheath or rod 30 (hereinafter referred to as a pusher member), typically made from a plastics material, is mounted coaxial with and radially outside of the inner catheter 24. The pusher member 30 is “thick walled”, that is the thickness of its wall is preferably several times greater than that of the guide wire catheter 24. In some instances, the pusher member 30 and the inner catheter 24 are the same component, possibly having different outer diameters at the location at which the stent 18 is to be carried.

A sheath 32 extends coaxially over and radially outside of the pusher member 30. The pusher member 30 and the sheath 32 extend distally to the manipulation region 12.

The implant 18, which may be a stent, a stent-graft or any other implant or prosthesis deliverable by this device 10, is retained in a compressed condition by the sheath 32. The sheath 32 extends proximally to a sheath manipulator and haemostatic sealing unit 34 of the external manipulation section 12. The haemostatic sealing unit 34 includes a haemostatic seal (not shown) and a side tube 36 held to the unit 34 by a conventional luer lock 38.

The sheath manipulator and haemostatic sealing unit 34 also includes a clamping collar (not shown) that clamps the sheath 32 to the haemostatic seal and a silicone seal ring (not shown) that forms a haemostatic seal around the pusher rod 30. The side tube 38 facilitates the introduction of medical fluids between the pusher rod 30 and the sheath 32. Saline solution is typically used.

During assembly of the introducer 10, the sheath 32 is advanced over the proximal end of the dilator tip 20 of the proximal attachment region 16 while the implant 18 is held in a compressed state by an external force. A suitable distal attachment (retention) section (not visible in this view) is coupled to the pusher rod 30 and retains a distal end 40 of the prosthesis 18 during the procedure. The distal end of the prosthesis 18 may be provided with a loop of material (not shown) through which a distal trigger wire 42 extends. The distal wire also extends through an aperture (not shown in FIGS. 1 and 2) in the proximal attachment section 40 into an annular region 44 between the inner catheter 24 and the pusher rod 30. The distal trigger wire 42 extends through the annular space 44 to the manipulation region 12 and exits the annular space 44 at a distal wire release mechanism 46.

A proximal portion of the external manipulation section 12 includes at least one release wire actuation section 50 mounted on a body 48, in turn mounted onto the pusher member 30. The inner catheter 24 passes through the body 48. The distal wire release mechanism 46 and the proximal wire release mechanism 50 are mounted for slidable movement on the body 48.

The positioning of the proximal and distal wire release mechanisms 46 and 50 is such that the proximal wire release mechanism or mechanisms 50 must be moved before the distal wire release mechanism 46 can be moved, such that the proximal end of the implant, that is the end of the implant which will be upstream in the direction of fluid flow in the patient's vasculature, is released first. Therefore, the distal end of the implant 18 cannot be released until a self-expanding zigzag stent thereof has been released. Clamping screws 52 prevent inadvertent early release of the prosthesis 18. A haemostatic seal (not shown) is included so that the release wires can extend out through the body 48 without unnecessary blood loss during the medical procedure.

A proximal portion of the external manipulation section 12 includes a pin vise 54 mounted onto the proximal end of the body 48. The pin vise 54 has a screw cap 56. When screwed in, vise jaws (not shown) of the pin vise 54 clamp against or engage the guide wire catheter 24. When the vise jaws are engaged, the inner catheter 24 can only move with the body 48 and hence it can only move with the pusher member 30. With the screw cap 56 tightened, the entire assembly can be moved together as one piece.

Once the introducer assembly 12 is in the desired deployment position, the sheath 32 is withdrawn to just proximal of the distal attachment section 14. This action releases the middle portion of the implant 18, in this example a stent or stent-graft, so that it can expand radially. Consequently, the stent or stent-graft 18 can still be rotated or lengthened or shortened for accurate positioning. The proximal end self-expanding stent however, is still retained at the dilator tip 16 by means of the release wires. Also, the distal end of the stent or stent-graft 18 will still retained within the sheath 32.

Next, the pin vise 54 is released to allow small movements of the inner catheter 24 with respect to the pusher rod 30 to allow the stent or stent-graft 18 to be lengthened, shortened, rotated or compressed for accurate placement in the desired location within the lumen. X-ray opaque markers (not shown) may be placed along the stent or stent-graft 18 to assist with placement of the prosthesis.

When the proximal end of the stent or stent-graft 18 is in place, the proximal trigger wire (not shown) is withdrawn by movement of the proximal wire release mechanism. The proximal wire release mechanism 50 and the proximal trigger wire can be completely removed by passing the proximal wire release mechanism 50 over the pin vise 54, the screw cap 56 and the connection unit 26.

Next, the screw cap 56 of the pin vise 54 is loosened, after which the inner catheter 24 can be pushed in a distal direction, that is towards the inside of the patient, so as to move the dilator tip 20 in a distal direction. This fully releases the proximal end of the stent or stent-graft 18, allowing it to expand so as to engage the lumen walls of the artery or vein. From this stage on, the proximal end of the stent or stent-graft 18 cannot be moved again.

Once the proximal end of the stent or stent-graft 18 is anchored, the sheath 32 is withdrawn distally of the proximal attachment section 14, which withdrawal allows the distal end of the stent or stent-graft 18 to expand. Until this point and in particular until the distal release mechanism 46 is actuated to release the distal trigger wires from the distal end of the stent 18, the distal may still be repositioned as needed.

FIG. 3 shows an embodiment of catheter assembly similar to the catheter assembly 18-28 of FIGS. 1 and 2. The catheter assembly includes an inner catheter or cannula 124 made of any suitable structure known in the art and having a length suitable for the medical application to which it is to be put to use, typically up to one and a half metres in length.

Attached at the proximal end 126 of the inner catheter 124 there is a fitting 128, typically of a rigid plastics material, for example polycarbonate. The fitting 128 includes a disc-like handle portion 130 for use by the surgeon and a threaded connector 132, usefully of a luer lock type.

The distal end 122 of the catheter assembly includes a dilator tip 20 and carries a stent, stent graft or other device 18 to be implanted, as with the catheter assembly of FIGS. 1 and 2.

An open bore (not shown in the Figures) extends within the catheter 124, the fitting 128 and the dilator tip 20, that is from the proximal end 126 to the distal end 122, for receiving a guide wire.

A stress relief collar or sleeve 134 (hereinafter referred to as a collar) fits over an end of the fitting 128 and also over a proximal end of the inner catheter. The stress relief collar 134 is made of a resilient material, such as silicon, silicone rubber or any of the other materials typically used for catheter strain relief sleeves. Other examples include nylon and Pebax™ (polyester bloc amide).

The collar 134 preferably has a tapering shape away from the fitting 128. This has the effect of providing support to the proximal end of the catheter 124 at the point of its junction with the rigid fitting 128. The tapering nature of the collar 134 also has the effect of providing a gradual transition in flexibility from the fitting 128 and catheter 124, thus minimising abrupt changes. This can prevent or substantially reduce the risk of kinking of the catheter 124 at its point of connection with the fitting 128, as is known in the art.

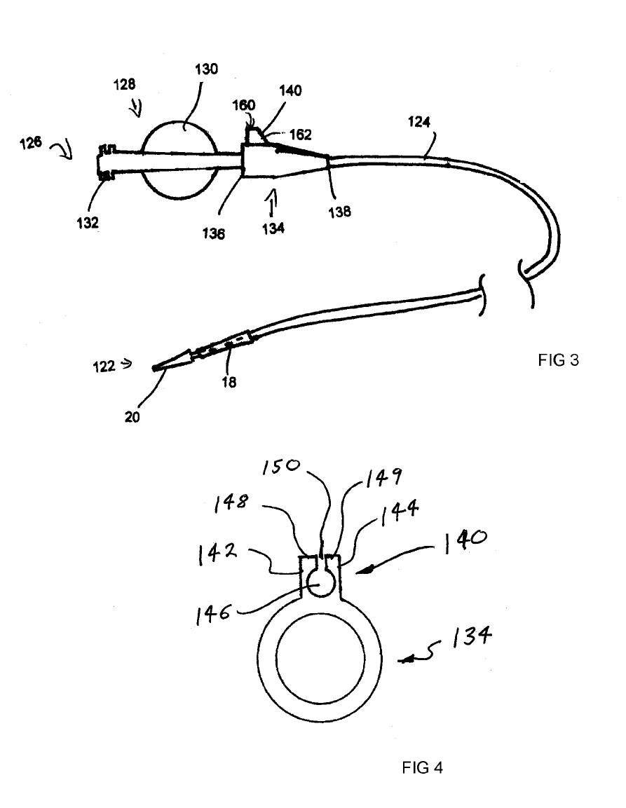

Referring also to FIG. 4, the stress relief collar 134 is also provided with a catheter holding clip 140 extending radially therefrom and integral therewith. In the preferred embodiment, the clip is formed integrally with the collar 134 and is of the same material. However, it is not excluded that the clip 134 might be formed of a different but nevertheless pliable material and that it may be attached to the collar after formation of these two components.

The clip 140 is formed of first and second facing fingers 142, 144 which in this embodiment are substantially parallel. At the free ends 148, 149 of the fingers 142, 144 there are provided enlarged feet elements facing one another and which are spaced from one another by a narrow elongate passage 150. At the bases of the fingers 142, 144 there is a space 146, preferably being round in cross-section as shown in FIG. 4. The space 146 is preferably wider than the passage 150. Advantageously, the passage 150 is slightly narrower than the diameter of the catheter 124. The free ends 148, 149 may have upper edges which are inwardly rounded or bevelled to present an inlet funnel feature for assisting in the insertion of a catheter into the clip.

Referring again to FIG. 3, in this embodiment the clip 140 has a proximal end 160 the free end of which is substantially parallel to the body of the collar 134 and a distal end 162 which tapers towards the collar 134.

Although FIG. 4 shows the clip 140 with a transverse dimension smaller than the diameter of the collar 134, it is envisaged that in some embodiments it could be the same, such that the side walls of the clip 140 extend directly from the widest part of the collar.

FIG. 5 shows the catheter 124 (of FIG. 3) coiled in two turns and having a section of each turn held within the clip 140. This is achieved by coiling the catheter 124 and pressing this through the passage 150 of the clip 140. In the case where the passage 150 is narrower than the diameter of the catheter 124, when the latter is pressed into the passage, the fingers 142, 144 deflect away from one another to widen the passage 150. As the fingers 142 and 144 are of a pliable material, that is of a soft material, this is achieved easily and without the need for much force. The round cross-section of the catheter 124 assists in opening the passage 150 and if desired the internal edges of the feet of the fingers 142, 144 could be bevelled to assist in this process.

The catheter section which is inserted through the passage 150, in this embodiment, comes to rest within the space 146 and is held there until is it pulled out by the surgeon or an assistant. The enlarged ends or feet of the fingers assist in retaining the catheter coil in the clip 134.

When it is desired to remove the catheter 124, each turn of coil is simply pulled out of the clip 124 by a force in a direction radially away from the collar 134. As the clip fingers 142, 144 are of a pliant material, this can be achieved easily and with little risk of damaging the catheter.

The difference in space and passage sizes shown in FIG. 4 is not essential for the performance of the clip 134. In some cases a catheter 124 could be held in a space between the fingers 142, 144 which is even throughout its length, assisted by the natural tendency of the catheter 124 to uncoil, by friction between the materials of the coil and clip or by a combination of such effects.

It is also envisaged that the fingers 142, 144 could provide a space of different shape to that shown in FIG. 4, for example widening gradually from the free ends 148, 149 of the fingers 142, 144 to their base at the body of the clip 134 and so on.

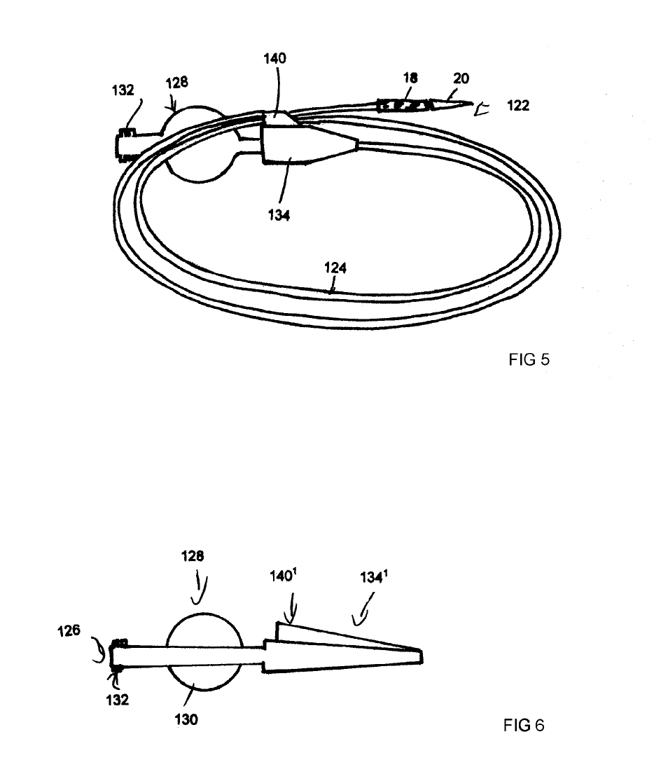

FIG. 6 shows another embodiment of fitting and collar assembly in which the collar 134′ is provided with a clip 140′ which extends substantially the whole length of the collar 134′ and which tapers for its entire extent. All other features of the clip 140′ and collar 134′ are as described herein.

The skilled person will appreciate that although the described embodiments relate to a clip integral with a stress relief collar, the invention is not limited to this implementation. The clip could equally be provided on a flexible sleeve provided for any purpose other than stress relief.

Although the specific embodiments which have been described relate to catheters for delivering stents, the teachings herein can be applied equally to catheters for other medical devices including an not limited to sent grafts, filters and occlusions devices. They can also apply to any other elongate medical element which can usefully be retained in a coiled form before and during their use. A guide wire assembly is one example of other medical component which could be used with a clip of the type taught herein.

1. A flexible fitting for a medical introducer assembly including an elongate collar made from resilient material and a clip element integral with the elongate collar and extending outwardly therefrom, the clip element being made from a material having substantially the same flexibility as the material of the elongate collar.

2. The flexible fitting according to claim 1, wherein the fitting is a stress relief fitting and the collar is a stress relief collar.

3. The flexible fitting according to claim 1, wherein the clip element is made from the same material as the elongate collar.

4. The flexible fitting according to claim 1, wherein the clip element includes first and second facing fingers providing therebetween a space for receiving a section of catheter.

5. The flexible fitting according to claim 4, wherein the fingers include at their ends remote from the collar an elongate opening for the passage of a section of catheter therethrough into the space.

6. The flexible fitting according to claim 4, wherein the fingers provide an elongate opening which is narrower than the space.

7. The flexible fitting according to claim 4, wherein the ends of the fingers are provided with enlarged facing feet members.

8. The flexible fitting according to claim 4, wherein the space is rounded in longitudinal cross-section.

9. The flexible fitting according to claim 4, wherein the fingers of the clip element taper towards the collar in the distal direction thereof.

10. The flexible fitting according to claim 1, wherein the clip element extends part way along the collar.

11. The flexible fitting according to claim 1, wherein the clip element is located on a proximal position of the collar.

12. (canceled)

13. (canceled)

14. The flexible fitting according to claim 1, wherein the clip element is formed of a material having a hardness on the Shore D scale of between 20 and 50.

15. A medical introducer assembly including an elongate introducer element, a fitting attached to the elongate introducer element, a flexible collar and a clip for holding one or more sections of the elongate introducer element, the clip being formed of a material having a flexibility no greater than a flexibility in curvature of the introducer element.

16. The medical introducer assembly according to claim 15, wherein the introducer is a catheter assembly.

17. The medical introducer assembly according to claim 15, wherein the collar is a stress relief collar for relieving stress differentials between the fitting and the elongate introducer element.

18. The medical introducer assembly according to claim 15, wherein the clip is substantially softer than the flexibility of the introducer element in radial compression.

19. The medical introducer assembly according to claim 15, wherein the clip is formed of a material having a hardness on the Shore D scale of between 20 and 50.

20. The medical introducer assembly according to claim 15, wherein the clip is formed of a material having a flexibility similar to that of the material of the collar.

21. The medical introducer assembly according to claim 15, wherein the stress relief collar is located over the elongate introducer element and connected to the fitting.

22. The medical introducer assembly according to claim 15, wherein the stress relief collar is located over a distal end of the fitting.

23. The medical introducer assembly according to claim 15, wherein the clip is integral with the collar.

24. A medical introducer assembly according to claim 15, wherein the clip is made of the same material as the collar.
